(19) United States
(12) Patent Application Publication
(10) Pub. No.: US 2026/0069561 A1
(43) Pub. Date: Mar. 12, 2026

(54) NEUROPROTECTIVE SUPPLEMENTS

(71) Applicant: Madhureeta Achari, MD, Houston, TX (US)

(72) Inventor: Madhureeta Achari, MD, Houston, TX (US)

(21) Appl. No.: 19/322,319

(22) Filed: Sep. 8, 2025

Related U.S. Application Data
(60) Provisional application No. 63/691,792, filed on Sep. 6, 2024.

(51) Int. Cl. A61K 31/198; A61K 31/14; A61K 31/4188; A61P 25/16; A61P 25/28

(52) U.S. Cl. CPC A61K 31/198; A61K 31/14; A61K 31/4188; A61P 25/16; A61P 25/28

(57) ABSTRACT

A supplement, and a method of treatment utilizing the supplement to elicit positive responses in a patient with neurological conditions is disclosed. The supplement comprises four components: N-acetylcysteine, glycine, choline, and biotin. In the preferred embodiment for treating adult patients, the effective supplement daily dosage comprises at least 1500 mg of glycine, 1500 mg of N-acetylcysteine, 1000 mg of choline, and 1000 μg of biotin. In another embodiment for treating pediatric patients, the supplement may comprise an effective supplement daily dosage of at least 500 mg of glycine, 500 mg of N-acetylcysteine, 250 mg of choline, and 250 μg of biotin. In a method embodiment, regular daily dosages of a supplement comprising over 1 g total of the four components are administered, resulting in improved outcomes among patients with cognitive impediments.

### CROSS REFERENCE TO RELATED APPLICATIONS

This application is a non-provisional application that claims priority to U.S. Provisional Application No. 63/691,792, entitled “Neuroprotective Supplements” filed Sep. 6, 2024.

### FIELD

The present disclosure relates, generally, to combinations of supplements that are commonly administered separately and in small doses. In particular, the present disclosure relates to the administration of, glycine, choline, and biotin in conjunction with each other, and in dosages beyond the recommended minimum.

### BACKGROUND

Many neurodegenerative conditions are known to be linked with deficient or excessive dietary habits, often due to these dietary habits resulting in chronic shortages of vital micronutrients, which in turn perform important roles in maintaining brain health.

NAC, herein N-acetylcysteine is often cited as having a therapeutic potential for the treatment of certain patients, as N-acetylcysteine is known to have powerful anti-inflammatory and anti-oxidant properties. Various clinical studies have explored the use of N-acetylcysteine as a part of a potential treatment for conditions like Parkinson's and Alzheimer's, as these studies have shown that N-acetylcysteine may possibly protect brain cells from oxidative stress and inflammation. It is to be noted that “Acetylcysteine” is the generic drug name known in the art to be used in hospitals and prescriptions, while “N-acetylcysteine (NAC)” is the same molecule, usually used in supplements and biochemical discussions. One of skill in the art would recognize these terms to be interchangeable.

Glycine is a non-essential amino acid that is involved in the production of gut chemicals, collagen, and most pertinently, neurotransmitters. It is also believed that glycine has anti-oxidant and anti-inflammatory properties, similar to N-acetylcysteine. Combinations of N-acetylcysteine and glycine are known to be used in supplements, because the two compounds synergize to promote glutathione, a peptide whose levels have been shown to diminish with age. Several clinical trials, which include the use of supplements containing N-acetylcysteine and glycine, have indicated possible neurological benefits in the patients taking the supplements and participating in the trials.

Choline is an essential amino acid that is required by the human body to produce the neurotransmitter acetylcholine, as well as to produce various other signaling structures and lipids for supporting structural integrity of the body's cell membranes and walls. Choline is most noted for its role in the removal of fat and cholesterol from the liver of the human body; however, choline has been known to play a vital role in brain development and some of the basic functions within the nervous system. This latter role of choline has led to clinical investigations for cognitive benefits in patients, although choline is rarely used in conjunction with the above-noted biotin combination of N-acetylcysteine and glycine.

Biotin (also known as vitamin B7) is an essential micronutrient, which is involved primarily as a cofactor for several enzymes responsible for energy production, fatty acid synthesis, and amino acid metabolism in the human body. Unlike the other three, biotin is most commonly known as a supplement for skin health, and not brain health. For example, in 2015, several initial clinical trials were conducted to explore the potential use of biotin for treating multiple sclerosis in patients; however, the studies ended without any notable success as any improvements largely failed to replicate.

As stated earlier, prior art supplement combinations have utilized some of these elements in certain limited combinations. So-called “GlyNAC” supplementation combining glycine and N-acetylcysteine is known in the art; an exemplar of these supplements can be found in US 2024/0041812 to Sekhar et al (a continuation of U.S. Pat. No. 10,952,982 to same), which discloses the use of a combination of N-acetylcysteine and glycine for various cognitive improvements.

Prior art disclosing combinations of the above with glycine and biotin appears to be substantially more rare, although some specialized multi-vitamin formulations may include all four. An example of such prior art is U.S. Pat. No. 10,105,419 to Holstein et al, which discloses a complex dietary supplement for the treatment of type II diabetes comprising all four compounds, in combination with over thirty (30) other compounds, as is typical in the prior art.

Because these complex multivitamins contain numerous other compounds, the vitamin supplements contain small overall dosages of the four specific compounds discussed above. For example, with regard to the Holstein supplement, the only example formulation in which glycine is present contains a total glycine dosage of 1250 mcg (1.25 mg). Moreover, the absolute and relative dosages can differ widely. For example, the proportions of glycine to N-acetylcysteine in the Sekhar supplement can be anywhere from 1 to 99%.

A need exists for a supplement that contains all four compounds, including N-acetylcysteine, glycine, choline and biotin, in sufficient dosage amounts to successfully treat patients suffering from neurodegenerative conditions, for achieving documented improvements in the patient's brain health and cognitive functions.

A need exists for a neuroprotective supplement which utilizes all four of the compounds in order to provide additional neuroprotective activity beyond what any one or two of the compounds are capable of in isolation, and which additionally provides the compounds at a dosage greater than broad-spectrum, multi-vitamins having over thirty (30) active components.

The disclosed supplement has produced unexpected and substantial positive outcomes in patients with neurodegenerative conditions, as confirmed by both objective neurological assessments and subjective reports from family members and caregivers. These results indicate that the combination and dosage levels of N-acetylcysteine, glycine, choline, and biotin, as disclosed herein, provide synergistic and enhanced neuroprotective effects beyond what is observed with prior multivitamin formulations. Embodiments and methods disclosed herein meet the aforementioned need by improving cognitive function, slowing disease progression, and enhancing quality of life. Documented benefits of the supplement include, but are not limited to: delaying the need for full-time caregiving-resulting in significant cost savings; reducing the incidence of complications that may elevate mortality risk (e.g., neurotoxic events or associated comorbidities); and enabling patients to maintain a higher level of independence, thereby reducing their overall burden on caregivers and the community.

### SUMMARY

The present disclosure describes a supplement, and a method of treatment utilizing the supplement, to elicit positive responses in patients with neurological conditions and preventive effects in patients that have not been formally diagnosed with a neurological condition. The supplement comprises four components: N-acetylcysteine, glycine, choline, and biotin.

In a method embodiment, regular daily dosages of a supplement comprising over 1 g total of the four components are administered, resulting in improved outcomes among patients with cognitive impediments.

In an embodiment, the glycine and the N-acetylcysteine are administered in an amount of at least 1500 mg per day.

In another embodiment, the glycine and the N-acetylcysteine are administered in an amount of at least 500 mg per day.

In an embodiment, the choline is administered in an amount of at least 250 mg per day.

In another embodiment the choline is administered in an amount of at least 500 mg per day.

In an embodiment, biotin is administered in an amount of at least 250 μg per day.

In another embodiment, the biotin is administered in an amount of at least 1000 μg per day.

In one embodiment, the method comprises testing the patient's blood to obtain baseline levels of biomarkers relevant to neurological health; analyzing the baseline biomarker levels to determine supplement needs; formulating a supplement comprising four components: glycine, N-acetylcysteine, choline, and biotin; administering the supplement in a daily dosage, wherein: glycine is present in a range of 500 mg to 5000 mg; N-acetylcysteine is present in a range of 500 mg to 5000 mg; choline is present in a range of 250 mg to 5000 mg; and, biotin is present in a range of 250 μg to 10000 μg.

In an embodiment, the daily dosage may be adjusted based on follow-up blood tests to optimize treatment outcomes.

In an embodiment, glycine is present in an amount between about 500 mg and about 5000 mg; N-acetylcysteine is present in an amount between about 500 mg and about 5000 mg; choline is present in an amount between about 250 mg and about 5000 mg; and, biotin is present in an amount between about 250 mg and about 10000 μg.

In an embodiment, the supplement is formulated for oral daily administration.

In an embodiment, supplement is administered as part of a personalized treatment protocol based on biomarker testing of the patient's blood.

In an embodiment, biomarkers comprise one or more of: inflammatory markers, oxidative stress indicators, or markers of neuronal injury.

In an embodiment, the total daily dosage comprises: about 1500 mg of glycine; about 1500 mg of N-acetylcysteine; about 1000 mg of choline; and, about 10000 μg of biotin.

In an embodiment, the supplement is used to treat or manage a neurological disorder selected from the group consisting of: neurodegenerative disease, traumatic brain injury, stroke, cognitive impairment, and neuroinflammation.

In an embodiment for treating adult patients, the effective supplement daily dosage comprises at least 1500 mg of glycine, 1500 mg of N-acetylcysteine, 1000 mg of choline, and 1000 μg of biotin.

In an embodiment for treating pediatric patients, the supplement may comprise an effective supplement daily dosage of at least 500 mg of glycine, 500 mg of N-acetylcysteine, 250 mg of choline, and 250 μg of biotin.

### BRIEF DESCRIPTION OF THE DRAWINGS

In the detailed description of the embodiments, presented below, reference is made to the accompanying drawings:

FIG. 1 is a table showing test data obtained from neurodegenerative patients while using the disclosed supplementation.

FIG. 2 is a graph showing the changes in memory facet scores for each neurodegenerative patient over a one year period during the administration of the disclosed supplementation.

FIG. 3 is a graph showing the changes in language facet scores for each neurodegenerative patient over a one year period during the administration of the disclosed supplementation.

FIG. 4 is a graph showing the changes in social interaction facet scores for each neurodegenerative patient over a one year period during the administration of the disclosed supplementation.

FIG. 5 is a graph showing the changes in anxiety facet scores for neurodegenerative each patient over a one year period during the administration of the disclosed supplementation.

FIG. 6 is a graph showing the changes in ADL facet scores for each neurodegenerative patient over a one year period during the administration of the disclosed supplementation.

FIG. 7 is a graph showing the changes in care giver stress facet scores for each neurodegenerative patient over a one year period during the administration of the disclosed supplementation.

FIG. 8 is a graph showing the changes in caregiver burden facet scores for each neurodegenerative patient over a one year period during the administration of the disclosed supplementation.

FIG. 9 is a graph showing the improvement in the cognitive score of a neurodegenerative patient after six months of supplementation.

FIG. 10 is a table showing test data obtained from non-neurodegenerative patients while using the disclosed supplementation.

FIG. 11 is a graph showing the changes in memory facet scores for each non-neurodegenerative patient over a one year period during the administration of the disclosed supplementation.

FIG. 12 is a graph showing the changes in energy facet scores for each non-neurodegenerative patient over a one year period during the administration of the disclosed supplementation.

### DETAILED DESCRIPTION OF THE EMBODIMENTS

Before describing selected embodiments of the present disclosure in detail, it is to be understood that the present invention is not limited to the particular embodiments described herein. The disclosure and description herein is illustrative and explanatory of one or more presently preferred embodiments and variations thereof, and it will be appreciated by those skilled in the art that various changes in the design, organization, order of operation, means of operation, equipment structures and location, methodology, and use of mechanical equivalents may be made without departing from the spirit of the invention.

As well, it should be understood the drawings are intended to illustrate and plainly disclose presently preferred embodiments to one of skill in the art but are not intended to be manufacturing level drawings or renditions of final products and may include simplified conceptual views as desired for easier and quicker understanding or explanation.

Because many varying and different embodiments may be made within the scope of the concept(s) herein taught, and because many modifications may be made in the embodiments described herein, it is to be understood that the details herein are to be interpreted as illustrative and non-limiting.

It is to be understood that this application is not limited to particular process and manufacturing parameters, as these may vary.

Whenever a numerical range with a lower limit and an upper limit is disclosed, any number and any included range falling within the range is specifically disclosed. In particular, every range of values (of the form, “from about a to about b” or, equivalently, “from approximately a to b” or, equivalently, “from approximately a-b”) disclosed herein is to be understood to set forth every number and range encompassed within the broader range of values.

In the description that follows, unless otherwise indicated the term “about” means±5%.

In an embodiment, the supplement for treating patients with neurological disorders and/or diseases may comprise four components: glycine, N-acetylcysteine, choline, and biotin.

In an embodiment, the dosage of glycine may range between 500 mg and 5000 mg.

In an embodiment, the dosage of N-acetylcysteine may range between 500 mg and 5000 mg.

In an embodiment, the dosage of choline may range between 250 mg and 5000 mg.

In an embodiment, the dosage of biotin may range between 250 μg and 10000 μg.

In an embodiment, the supplement for treating pediatric patients with neurological disorders and/or diseases comprises a daily administration of at least 500 mg of glycine and N-acetylcysteine, respectively, at least 250 mg of choline and at least 250 μg of biotin.

In another embodiment, the dosages of the supplement, for treating adult patients with neurological disorders and/or diseases may be elevated and administered as follows: 1500 mg of glycine and N-acetylcysteine, respectively, 2000 μg of biotin, and 1000 mg of choline.

The total daily dosage of the supplement is formulated to be administered orally in six (6) discrete dosage units, such as pills, capsules, or tablets. The total effective dosage amount is divided equally among the six units to ensure consistent bioavailability and therapeutic effect throughout the day.

To form supplement dosages suitable for optimal use in patients, requirements of the disclosed supplement may be tailored based on individual patient needs, which can be determined through diagnostic testing, including but not limited to, blood-based testing. Blood tests may be used to assess baseline and ongoing levels of key metabolites, vitamins, and related compounds and relevant biomarkers, including inflammatory markers, oxidative stress indicators, and markers of neuronal injury. These clinical metrics can guide personalized dosage adjustments to optimize therapeutic efficacy and minimize potential side effects.

For example, patients exhibiting low plasma levels of choline or elevated homocysteine may require increased choline supplementation to support methylation and neurotransmitter synthesis. Similarly, suboptimal levels of biotin-dependent enzyme activity or glutathione depletion may necessitate adjustments in biotin or N-acetylcysteine dosage, respectively. This individualized approach allows for a dynamic approach of one or more components of the supplement to achieve target therapeutic levels while accommodating differences in metabolism, absorption, disease severity, and treatment response across patient populations.

In a preferred embodiment, it is generally recommended that the subject (e.g., a human patient) space the administration of the dosage units at regular intervals over the course of a 24-hour period to maintain stable plasma concentrations of the active ingredients. For example, the subject may administer the dosage by taking three (3) pills twice daily (e.g., morning and evening) or two (2) pills three times daily (e.g., morning, afternoon, and evening), depending on individual tolerance, clinical indication, or practitioner guidance.

However, the dosing schedule may be adjusted to optimize pharmacokinetics, therapeutic effect, or patient compliance, and such variations are considered within the scope of the present disclosure.

The inventor has observed and documented, through numerous case studies, that the treatments described herein produce remarkable, unexpected improvement, and often dramatic progress, or at least a stabilization, in the functionality of the patients previously diagnosed with long-term declines due to neurological impairment.

A set of thirty-six case studies were conducted to investigate the effectiveness of the disclosed supplementation. The case studies can be divided into two groups.

The first group of eleven case studies includes patients that have been formally diagnosed with neurodegenerative conditions by a qualified medical professional. The neurodegenerative conditions diagnosed include Severe Alzheimer's Disease (“AD (Sev)”), Moderate Alzheimer's Disease (“AD (Mod)”), Mild Alzheimer's Disease (“AD (Mild)”), Aphasia, Primary Progressive Aphasia (“PPA”), Mild Cognitive Impairment with confirmed Alzheimer's pathology (“MCI w/ AD”), Mild Cognitive Impairment without confirmed Alzheimer's pathology (“MCI w/o AD”), and Parkinson's Disease (“PD”).

The second group includes patients who have not received a formal clinical diagnosis of a neurodegenerative condition by a qualified medical professional, but who have nonetheless presented with subjective cognitive complaints, such as memory-related concerns. These individuals may have reported symptoms such as forgetfulness, difficulty concentrating, or trouble recalling recent events. Additionally, members of this group may possess one or more risk factors that are associated with the potential future development of neurodegenerative diseases. Such risk factors include, but are not limited to, a documented family history of Alzheimer's Disease, Parkinson's Disease, or other related neurodegenerative conditions. Importantly, while these individuals may not meet the clinical criteria for diagnoses of one of the aforementioned neurodegenerative conditions, they represent a population of interest for early intervention, screening, or longitudinal study due to their potential predisposition or early-stage manifestation of cognitive decline. Accordingly, this group may be referred to as a “preclinical,” “at-risk,” or “subjective cognitive decline” population. Individuals in this second group are intended to receive the disclosed supplementation as a preventative measure, with the goal of mitigating the risk or delaying the onset of neurodegenerative symptoms or conditions. The supplementation is administered to promote cognitive resilience, support neurological function, and potentially alter the progression or emergence of disease-related pathology in high-risk or symptomatic-but-undiagnosed individuals.

For each case study, a baseline evaluation protocol comprising a neurological history assessment and a neurological examination was conducted. The neurological history evaluation comprised gathering patient information, including, but not limited to, the patient's chief complaint and current symptoms, history of present illness, review of organ systems, past medical and surgical history, current and historical medication used, allergies and sensitivities, family and social history, and other subjects as identified during the evaluation. The neurological examination evaluation comprised examining the patient's mental state, heart and neck, cranial nerves, motor system, cerebellar function, gait and stance, spine, and other body systems and functions identified during evaluation.

Each case study patient was prescribed the following supplement dosages to be administered: 2000 μg biotin, 1000 mg choline, 1500 mg Glycine, and 1500 mg N-acetylcysteine.

Representative case study examples showing the efficacy and unexpected results of patients from group 1, i.e., patients who have been formally diagnosed with a neurodegenerative are described below.

### Case Study 1

Case study 1 is a 71-year-old white male who presented with cognitive and speech impairments beginning in 2017. The patient underwent formal neuropsychological testing and was subsequently diagnosed with primary progressive aphasia in 2022. In November 2023, the patient began a supplementation routine in accordance with the embodiments disclosed above.

Within months, the patient's family reported a drop in confusion and a rise in the patient's focus. A neurological exam in March 2024, showed improved mood and engagement in verbal responses, and the patient was able to speak in complete sentences, with rapid speech. Formal neuropsychological testing performed in April 2024, showed an improvement in a neurological clock-drawing test. The neurological clock drawing test is used to assess cognitive functions, including memory, visual-spatial skills, and executive functions. The standard clock drawing test is scored on 10-point scoring system and points are assigned for a correctly drawn clock face, the placement of numbers, and the accurate positioning of clock hands to a requested time. Errors in the drawing, such as incorrect number placement, distorted shapes, or improper hand position, can indicate various neurological conditions. A score of 10 indicates a perfect score with correct clock details.

After one year of treatment, the patient showed substantial improvement from three out to ten score to a six out of ten. A score of three out of ten may missing clock hands and indicates cognitive impairment. A score of six may include numbers inappropriately depicted or crowded indicates cognitive decline. Here, the patient also showed improvement in complex figure-drawing from deficient to borderline. The improvements were sustained for a period of at least eight (8) months.

The patient's family reported improvement with the patient's ability to interact well in social settings and travel. This is an improvement over past behavior and provides societal benefits as the patient is able to participate more fully with family, friends, and community, which is a significant positive effect of the disclosed supplement.

A neurological examination in August 2025, showed an improvement in the patient's ability to follow verbal commands and an increased willingness to participate in conversational engagement.

### Case Study 2

Case Study 2 is an 81-year-old white female who presented with memory issues in 2018. She was tested and diagnosed with Alzheimer's Disease, confirmed via amyloid Positron Emission Tomography (PET) scan in June 2021. The patient progressed slowly, developing partial seizures with secondary generalization in 2022, and she required full-time care by 2023. In November 2023, the patient began a supplementation routine in accordance with the embodiments disclosed above.

As a result of the patient's participation in this supplementation routine during the past 10 months, the patient's family has noted and reported a subjective improvement in her condition. For example, the patient was able to recall significant events in her life and to supply details on her own. She was able to attend her own 80^{th} birthday celebration, when previously she had been too agitated to attend social gatherings, including symphonies or lunches with friends. On examination in February 2024, she was able to state her children's names, and she was able to follow commands well, displaying improvement from her previous evaluation. These improvements have been sustained.

After one year of treatment with the disclosed supplementation, the patient's memory for recognizing caregivers and family improved. The patient was able to recall her husband's date of birth and their anniversary, with regularity. The patient's agitation declined significantly. Social interactions also improved. Notably, the patient was able to attend and dance at a wedding.

### Case Study 3

Case Study 3 is an 81-year-old white female with early-onset Alzheimer's Disease. In December 2023, the patient began a supplementation routine in accordance with the embodiments disclosed above.

By April 2024, both her family and primary care provider noted an improvement in her condition. The patient was more active and able to cook for herself, which she had not been able to do at all prior to commencing this supplementation treatment. These improvements have been sustained.

After one year of treatment with the disclosed supplementation, the patient has remained stable. The patient showed reduction in both frustration and anxiety. As a result, the patient was able to move to an independent living facility. Importantly, with the supplementation, the patient was able to find her way around the facility with no disorientation.

### Case Study 4

Case Study 4 is a 78-year-old white male with mild cognitive impairment, progressing to Alzheimer's Disease. His family said he was becoming more withdrawn and irritable. In January 2024, the patient began a supplementation routine in accordance with the embodiments disclosed above.

Evaluation in July 2024 showed his memory and activities were stable. The patient was able to attend parties for much longer, and he was able to dance and enjoy his company, whereas before he would become agitated and isolate himself. These improvements have been sustained.

The patient was diagnosed with Alzheimer's Disease using an amyloid PET scan. After one year of supplementation, the patient remains independent with activities of daily living. Some of these activities include mowing the lawn and walking his dog. The patient's mood also improved. Moreover, the patient showed increased interaction with family.

### Case Study 5

Case Study 5 is a 77-year-old Indian female diagnosed with early Alzheimer's Disease in October 2023, with progressive memory loss. In November 2023, the patient began a supplementation routine in accordance with the embodiments disclosed above.

On evaluation in April 2024, the patient stated that she felt less forgetful, and she realized how bad her condition had been. Her family stated that they felt her condition had not deteriorated further since starting the supplementation treatment. Further, the patient resumed reading and note-taking, and she was able to independently recall events that she had read about. These improvements have been sustained.

After one year of treatment with the disclosed supplementation, the patient remained independent in activities of daily living. Particularly the patient is able to effectively use her smartphone and computer, and is actively preparing to write her second book. The patient showed improved interaction with family and friends.

### Case Study 6

Case Study 6 is a 90-year-old white male with mild cognitive impairment. He had retired and stopped all work due to forgetfulness. In November 2023, the patient began a supplementation routine in accordance with the embodiments disclosed above.

On evaluation in December 2023, the patient stated that he had resumed part-time work, seeing clients and doing insurance analyses for his company. In addition, he was able to attend and speak at a two-hour meeting. This patient reported a substantial increase in energy and focus. These improvements have been sustained.

After one year of treatment with the disclosed supplementation, the patient continued to work, travel, and care for his wife, who has multiple medical issues. The patient reported a stable memory and overall healthy feeling while using the supplement disclosed herein.

### Case Study 7

Case Study 7 is a 73-year-old white female with Parkinson's Disease. In December 2023, the patient began a supplementation routine in accordance with the embodiments disclosed above.

On evaluation in February 2024, she reported improvements in sleep, mood, and cognitive focus. These improvements have been sustained.

After one year of treatment with the disclosed supplementation, the patient continued to function well. In particular, the patient sustained the ability to travel on her own.

The unexpected benefits shown in case studies 1-7 include, but are not limited to, reduced anxiety and improved social interaction. These unexpected benefits provide a significant reduction to caregiver stress and burden, cost savings, and an overall improved family and community interaction. The ability of patients to participate more fully with family, friends, and community with reduced agitation provides a unique benefit and beneficial outcome associated with the disclosed supplementation. The disclosed supplementation is a nontoxic alternative that allows the patient to stay at home with family as the course of neurological illness progresses. As a result, it is an improvement over existing pharmacological medications used to manage anxiety in neurological patients with memory disorders, specifically existing medications that cause sedation and carry an increased risk of death.

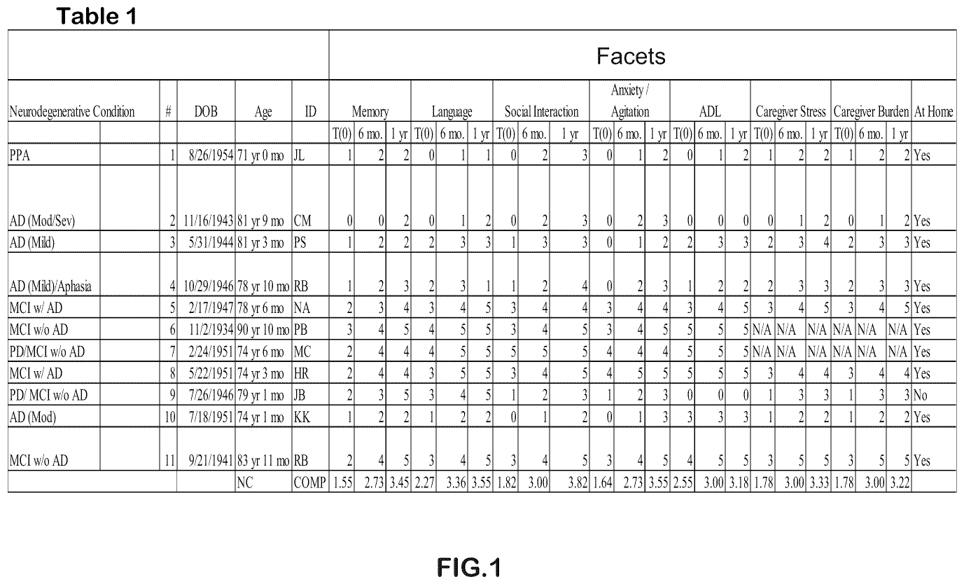

Referring now to FIG. 1, Table 1 is representative of test data obtained from examinations neurodegenerative patients while using the disclosed supplementation. To gather the data, a mental status examination is divided into a set of seven evaluative facets. These facets include memory, language, social interaction, anxiety, agitation, Activities of Daily Living (“ADL), caregiver stress, and care giver burden. The facet scores range from 0-5 based on a degree of degradation, where a score of 0 indicates severe degradation, and a score of 5 indicates no degradation. The scoring rubric is detailed in Table 2 below.

TABLE 2

| | |
|---|---|
| Not Degraded | 5 |
| Minimally Degraded | 4 |
| Mildly Degraded | 3 |
| Moderately Degraded | 2 |
| Highly Degraded | 1 |
| Severely Degraded | 0 |

This scoring system enables a quantifiable assessment of neurological and behavioral changes in response to the supplementation, allowing for comparative analysis over time or across patient cohorts.

The composite facet scores are derived from a comprehensive mental status evaluation that incorporates a number of clinical elements, including, but not limited to: appearance, behavior, mood, affect, speech, motor activity, thought process, thought content, perception, and cognition. Cognitive assessment components may further include alertness, orientation/sensorium, attention and concentration, memory (short-term and long-term), abstract reasoning, insight, and judgment of the medical professional.

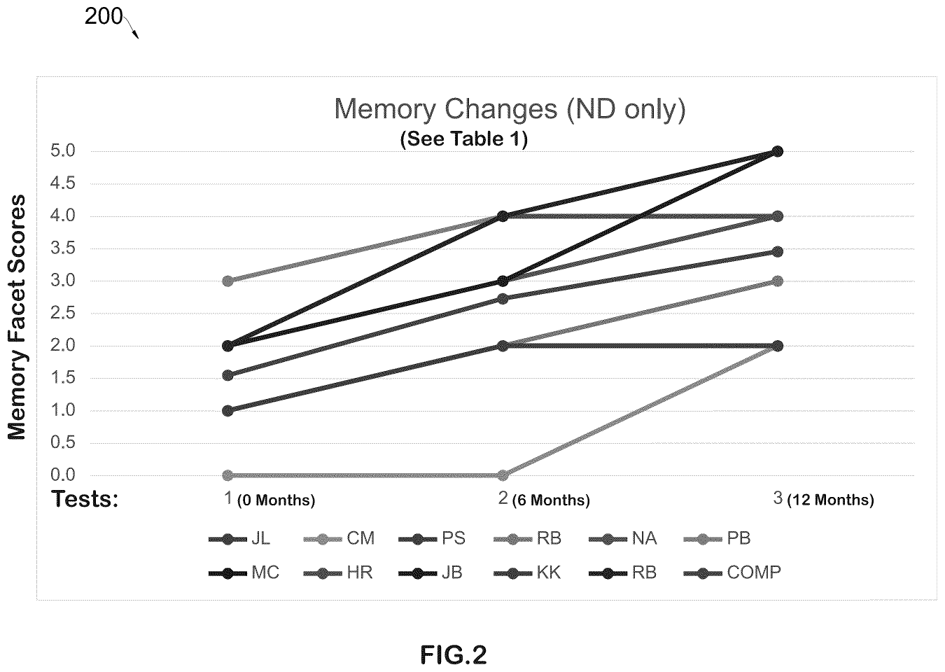

Referring to FIGS. 2-9, Graphs 200-900 each include data lines for patients that have been formally diagnosed with a neurodegenerative condition by a qualified medical professional. The patient data lines represent patient facet scores extrapolated from FIG. 1, Table 1, which have been recorded and plotted for each facet throughout the study duration, i.e., one year.

Referring now to FIG. 2, Graph 200 illustrates the changes in memory facet scores for each patient over one year period during the administration of the disclosed supplementation. As shown, graph 200 demonstrates a trend of progressive improvement in memory scores across all patients, indicating a positive cognitive response to the disclosed supplementation. The upward trajectory of the data lines reflects consistent improvement in memory function, as measured by the mental status examination criteria previously described. The observed data supports the efficacy of the supplementation in improving memory-related cognitive performance over time. Notably, the total average composite score for all patients over the one-year period of time improved from about 1.55 at baseline to about 3.45 at the end of the period.

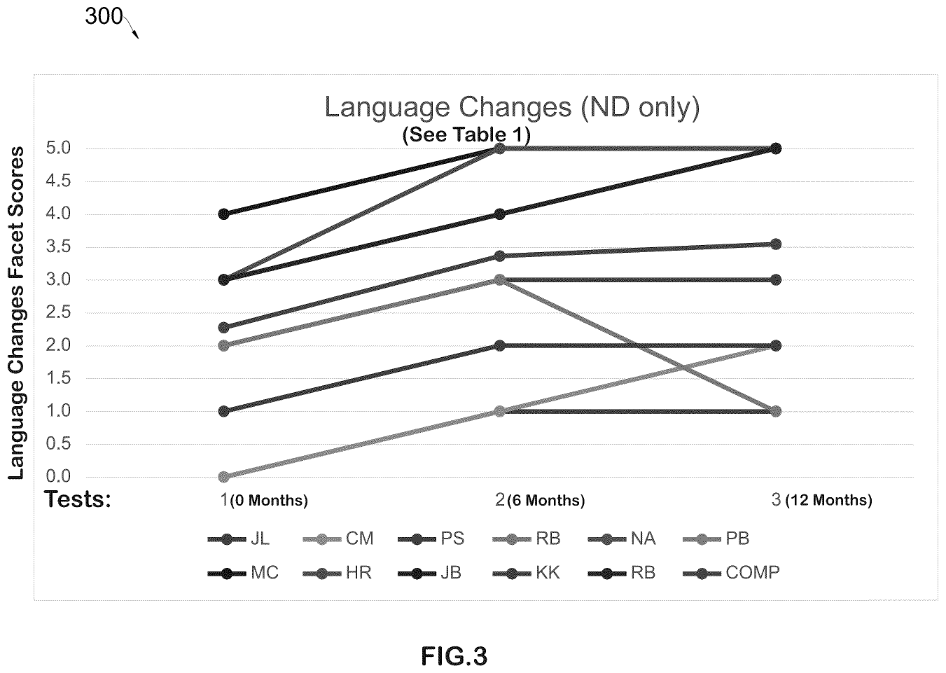

Referring now to FIG. 3, Graph 300 illustrates the changes in language facet scores for each patient over a one-year period during the administration of the disclosed supplementation. As shown in Graph 300, the overall average upward trajectory of the data lines indicates a consistent improvement in language function, as assessed by the mental status examination criteria previously described. The observed data supports the efficacy of the supplementation in improving language-related cognitive performance over time. Importantly, the total average composite score for all patients in the language facet over the one-year period of time improved from about 2.27 at baseline to about 3.55 at the end of the period.

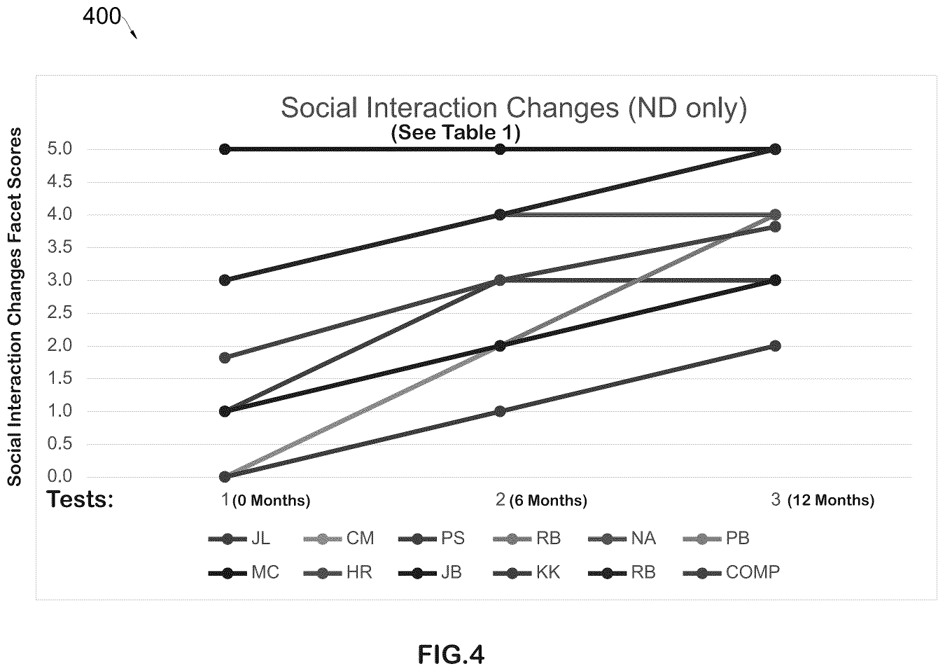

Referring now to FIG. 4, Graph 400 illustrates the changes in social interaction facet scores for each patient over a period of one year during the administration of the disclosed supplementation. The overall average upward trajectory and/or stabilization of the data lines shown in Graph 400 indicates a consistent improvement in social interaction function, as assessed by the mental status examination criteria previously described. The observed data supports the efficacy of the supplementation in improving social interaction-related performance over time. Importantly, the total average composite score for all patients in the social interaction facet over the one-year period of time improved from about 1.82 at baseline to about 3.82 at the end of the period.

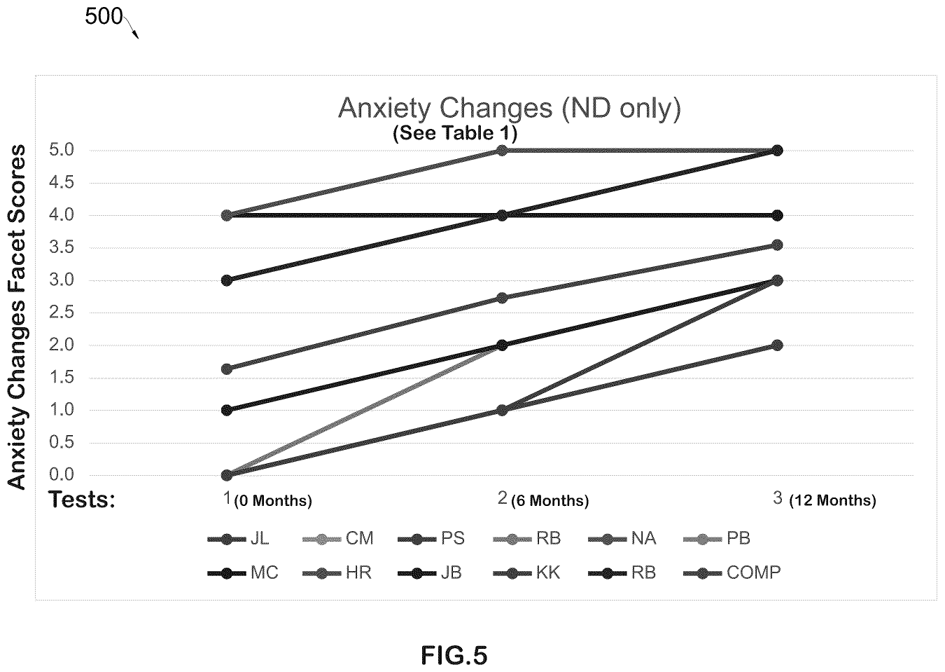

Referring now to FIG. 5, Graph 500 illustrates the changes in anxiety facet scores for each patient over a period of one year during the administration of the disclosed supplementation. The upward trajectory and/or stabilization of the data lines indicates a consistent improvement in the change of each patients' anxiety, as assessed by the mental status examination criteria previously described. The observed data supports the efficacy of the supplementation in improving anxiety-related cognitive performance over time. Importantly, the total average composite score for all patients in the anxiety facet over the one-year period of time improved from about 1.64 at baseline to about 3.55 at the end of the period.

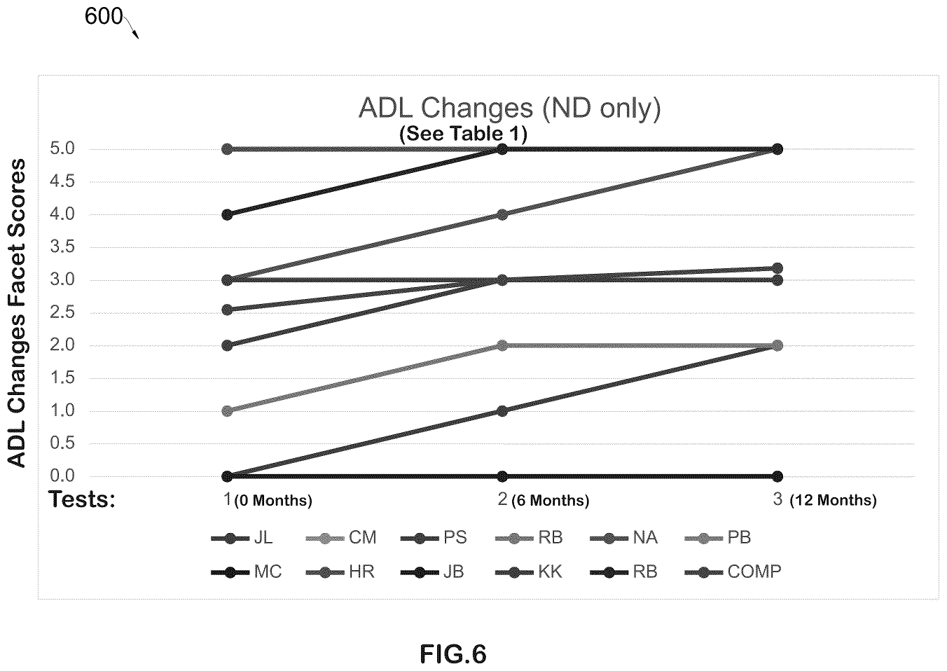

Referring now to FIG. 6, Graph 600 illustrates the changes in ADL facet scores for each patient over a period of one year during the administration of the disclosed supplementation. The upward trajectory and/or stabilization of the data lines indicates a consistent improvement in anxiety, as assessed by the mental status examination criteria previously described. The observed data supports the efficacy of the supplementation in improving ADL-related cognitive performance over time. Importantly, the total average composite score for all patients in the ADL facet over the one-year period of time improved from about 2.55 at baseline to about 3.18 at the end of the period.

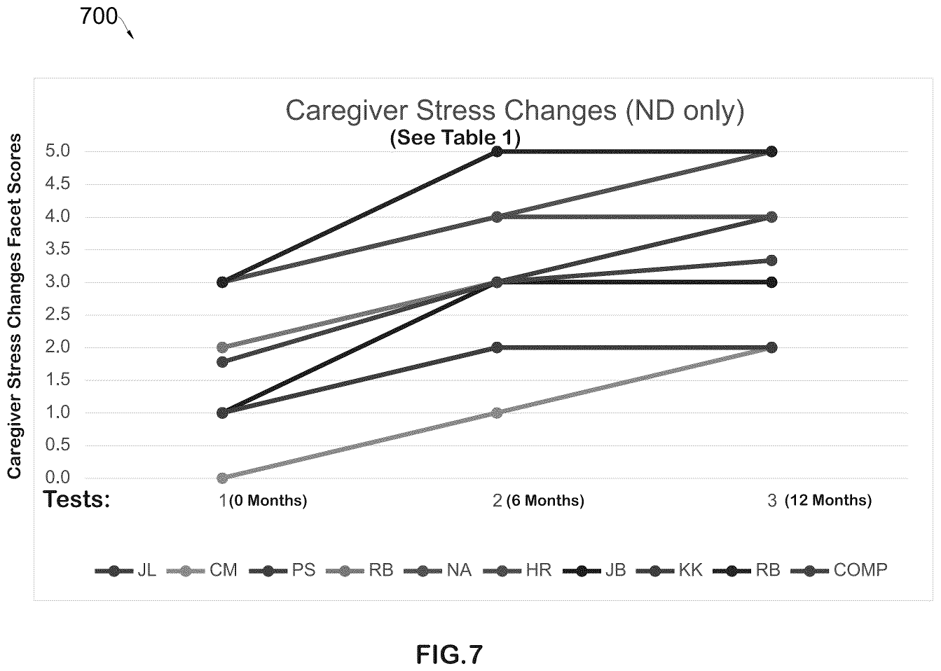

Referring now to FIG. 7, Graph 700 illustrates the changes in care giver stress facet scores for each patient over a period of one year during the administration of the disclosed supplementation. As shown, Graph 700 demonstrates a trend of progressive improvement in caregiver stress scores across all patients, indicating a positive cognitive response to the disclosed supplementation. The upward trajectory of the data lines reflects consistent improvement in the stress of having to rely on caregivers, as measured by the mental status examination criteria previously described. The observed data supports the efficacy of the supplementation in improving caregiver stress over time. Notably, the total average composite score for all patients over the one-year period of time improved from about 1.78 at baseline to about 3.33 at the end of the period.

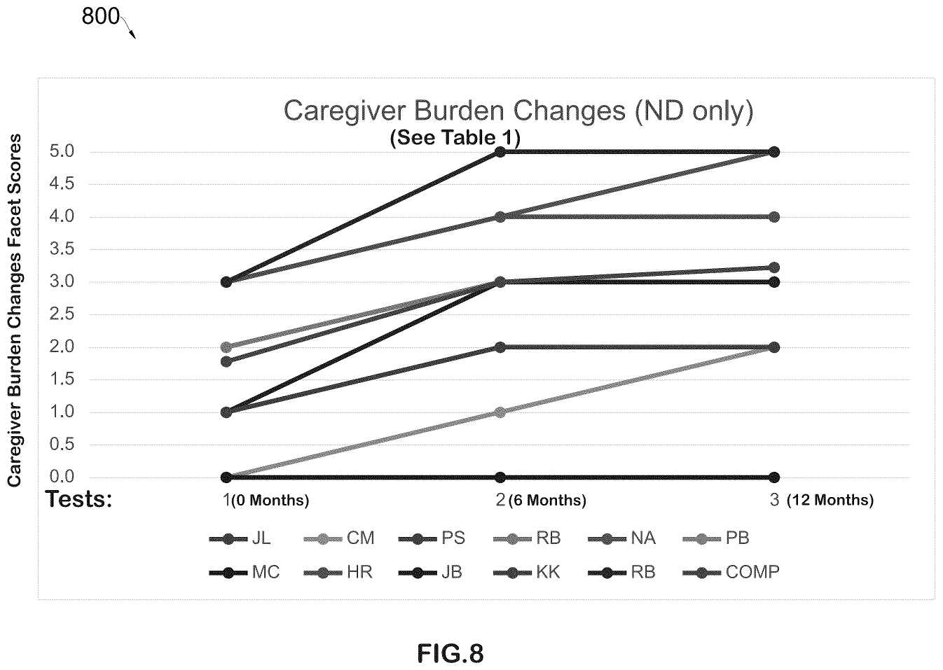

Referring now to FIG. 8, Graph 800 illustrates the changes in caregiver burden facet scores for each patient over a period of one year during the administration of the disclosed supplementation. The upward trajectory and/or stabilization of the data lines indicates a consistent improvement in regarding the burden of needing a caregiver, as assessed by the mental status examination criteria previously described. The observed data supports the efficacy of the supplementation in improving caregiver burden over time. Notably, the total average composite score for all patients in the caregiver burden facet over the one-year period of time improved from about 1.78 at baseline to about 3.22 at the end of the period.

In use, the data disclosed above and shown in FIGS. 1-8 support the efficacy of the disclosed supplementation. These improvements were observed in patients previously diagnosed with progressive neurological impairments, representing unexpected and clinically meaningful outcomes. Therefore, the disclosed supplementation can be seen to support the improvement and/or stabilization of multiple cognitive and functional domains over time.

Representative case study examples showing the efficacy and unexpected results of patients from group 2, i.e., patients who have not received a formal clinical diagnosis of a neurodegenerative condition by a qualified medical professional, but who have nonetheless presented with subjective cognitive complaints, are described below.

### Case Study 8

Case Study 8 is a 54-year-old white male with no formal neurological diagnosis, who had a seizure disorder, which had been controlled with medication. In November 2023, the patient began supplementation in accordance with the embodiments disclosed above.

On evaluation in April 2024, the Computerized Neurocognitive Status Vital Signs (CNSVS) cognitive testing showed an improvement of the patient by two (2) standard deviations in multiple areas, and that the patient had improved to 100% on all domains tested, as compared to a CNSVS test taken in 2023. These improvements have been sustained.

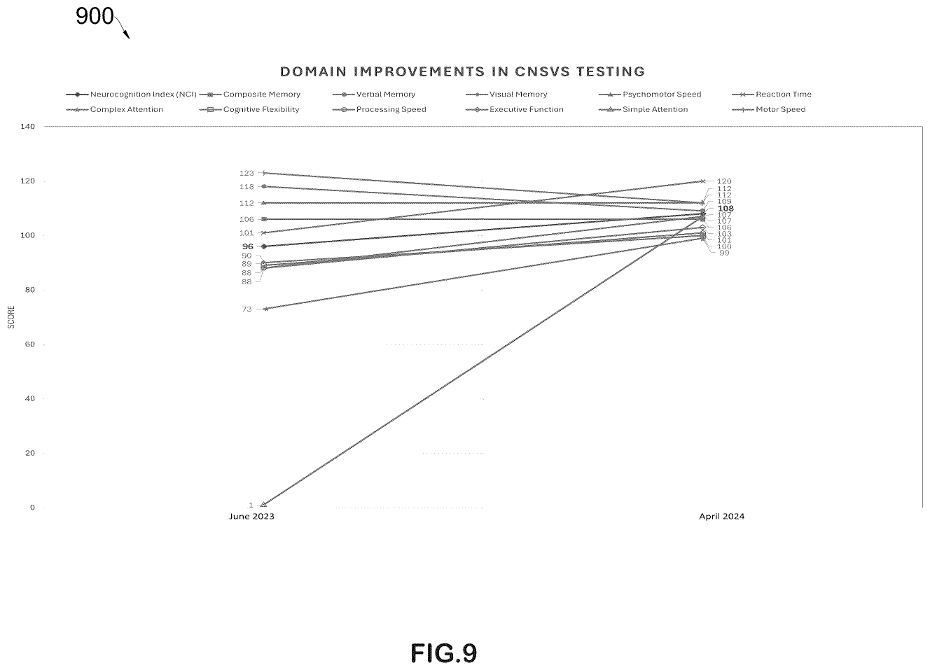

The improvement of Case Study 8 relative to the 2023 baseline is charted as FIG. 9. Referring to FIG. 9, in Graph 900 eight of the twelve domain scores improved after eight months of testing, with an overall Neurocognition Index score of 96 (40th percentile) improving to 108 (70^{th} percentile), including dramatic improvements in simple and complex attention (from 1^{st} and 4^{th} percentiles to 68^{th} and 47^{th} percentiles, respectively).

After one year of treatment with the disclosed supplementation, the patient-maintained improvement in all domains of testing and feels his mood and memory are improved. As a result, the patient believes he is able to function at a higher level in his occupation.

### Case Study 9

Case Study 9 is a 70-year-old white male with no formal neurological diagnosis, and is a retired attorney. His neurological history includes a stroke from which he had a complete recovery. In November 2023, the patient began a supplementation routine in accordance with the embodiments disclosed above. However, in April 2024, the patient discontinued the routine.

On evaluation in July 2024, the patient's spouse noted a significant difference in the functioning of her husband. During the time that her husband had followed the supplementation treatment, she noted that his functional improvements included more focus and motivation, and he experienced less forgetfulness. However, she stated that all of these functional improvements ceased, soon after the patient ceased his supplementation treatment.

After one year of treatment with the disclosed supplementation, the patient works and functions at a higher level than was reported before supplementation.

### Case Study 10

Case Study 10 is a 72-year-old white male with no formal neurological diagnosis, with a well-controlled seizure disorder and some peripheral neuropathy. In December 2023, the patient began a supplementation routine in accordance with the embodiments disclosed above.

On evaluation in January 2024, the patient discussed noticeable improvements in his energy, cognitive focus, and mood.

After one year of treatment with the disclosed supplementation, the improvements in cognitive function mood and energy continued. The patient reported a higher level in memory recall than peers of his same age.

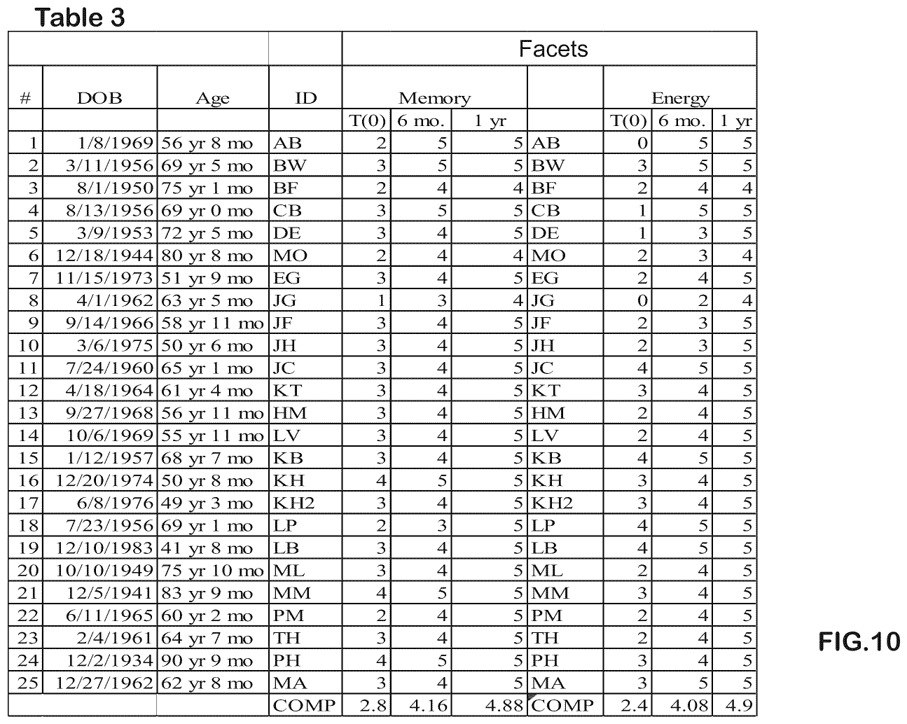

Referring now to FIG. 10, Table 3 presents representative test data obtained from examinations of all patients who have not received a formal clinical diagnosis of a neurodegenerative condition, but who have been prescribed the disclosed supplementation for its potential preventative effects. Because these individuals may not exhibit the need for a caregiver, significant language deficiencies, or changes in social interaction, the inventor has determined that the memory facet serves as the most appropriate metric for assessing outcomes and identifying potential unexpected improvements.

Like patients who have been diagnosed with neurodegenerative conditions, the non-neurodegenerative patient group was evaluated on a scale of 0-5, with each composite score derived according to the same rubric described in Table 2, based on a mental status evaluation based on a cognitive and energy assessment including, but not limited to, alertness, orientation/sensorium, attention and concentration, memory (short-term and long-term), abstract reasoning, mood, appearance, behavior, insight, and judgment of the medical professional.

This scoring system enables a quantifiable assessment of the prevention or delay of neurological degradation in response to the supplementation, allowing for comparative analysis over time and across patient cohorts.

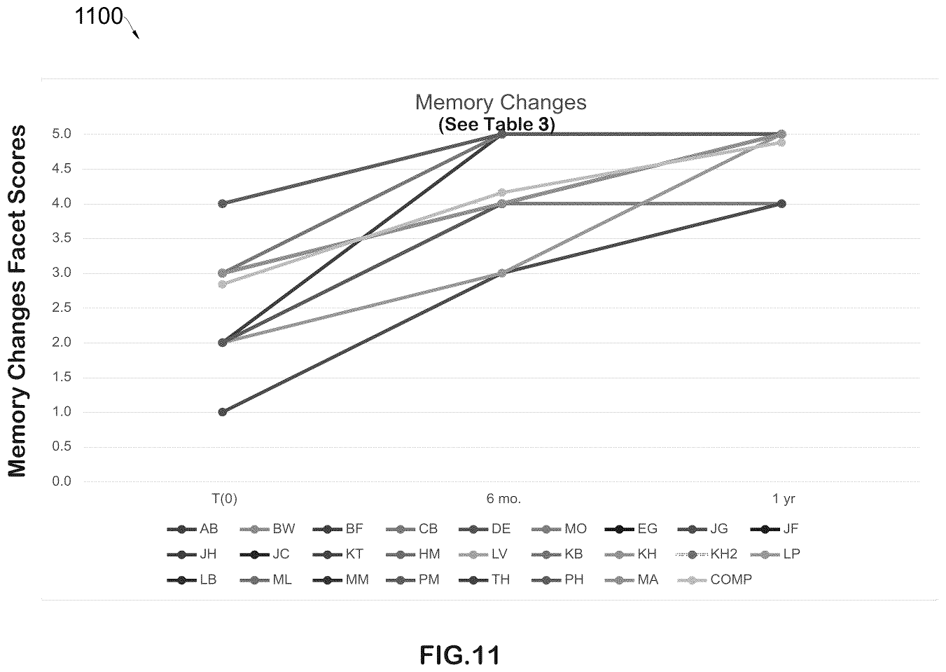

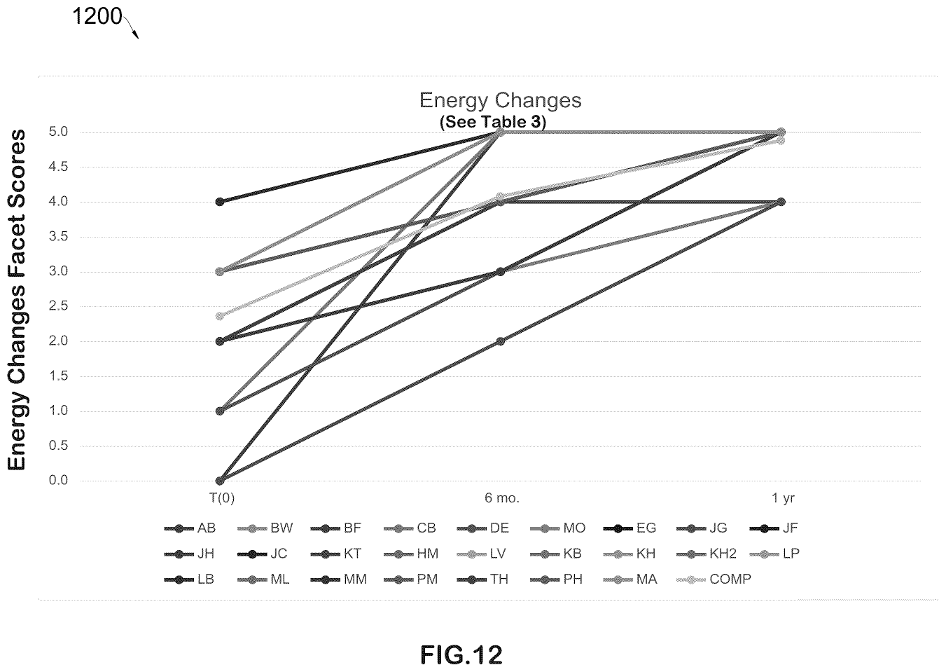

Referring to FIGS. 11-12, Graphs 1100 each include data lines for patients that have been formally diagnosed with a neurodegenerative condition by a qualified medical professional. The patient data lines represent patient facet scores extrapolated from FIG. 10, Table 3, which have been recorded and plotted for each facet throughout the study duration, i.e., one year.

Referring now to FIG. 11, Graph 1100 illustrates the changes in the memory facet score for each patient over a period of one year during the administration of the disclosed supplementation. The upward trajectory and/or stabilization of the data lines indicates a consistent improvement and prevention in memory degradation over time. The observed data supports the efficacy and preventative effects of the supplementation in improving memory-related cognitive performance over time. Notably, the total average composite score for all patients in the memory facet over the one year period of time improved from about 2.8 at baseline to about 4.88 at the end of the period.

Referring now to FIG. 12, Graph 1200 illustrates an energy change facet score for each patient over a period of one year during the administration of the disclosed supplementation. The upward trajectory and/or stabilization of the data lines indicates a consistent energy improvement and prevention of energy degradation over time. The observed data supports the efficacy and preventative effects of the supplementation in improving energy-related cognitive performance over time. Notably, the total average composite score for all patients in the memory facet over the one year period of time improved from about 2.4 at baseline to about 4.9 at the end of the period.

In use, the data disclosed above and shown in FIGS. 10-12 support the efficacy of the disclosed supplementation. Although these improvements were observed in patients who were not previously diagnosed with progressive neurological impairments, the data represents unexpected and clinically meaningful outcomes related to the preventative efficacy of the disclosed supplementation. Therefore, the disclosed supplementation can be seen to prevent the degradation of multiple cognitive and functional domains over time.

What is claimed is:

1. A method of sustaining and recovering neurological function, wherein the method comprises a daily administration of a supplement having at least four components, the four components comprising: glycine, N-acetylcysteine, choline, and biotin.

2. The method of claim 1, wherein the glycine and the N-acetylcysteine are administered in an amount of at least 1500 mg per day.

3. The method of claim 1, wherein the glycine and the N-acetylcysteine are administered in an amount of at least 500 mg per day.

4. The method of claim 1, wherein the choline is administered in an amount of at least 250 mg per day.

5. The method of claim 1, wherein the choline is administered in an amount of at least 500 mg per day.

6. The method of claim 1, wherein the biotin is administered in an amount of at least 250 μg per day.

7. The method of claim 1, wherein the biotin is administered in an amount of at least 1000 μg per day.

8. A method of sustaining and recovering neurological function, wherein the method comprises:
testing the patient's blood to obtain a set of baseline biomarker levels relevant to neurological health;
analyzing the set of baseline biomarker levels to determine supplement needs;
formulating a supplement comprising four components: glycine, N-acetylcysteine, choline, and biotin;
administering the supplement in a daily dosage, wherein:
glycine is present in a range of 500 mg to 5000 mg;
N-acetylcysteine is present in a range of 500 mg to 5000 mg;
choline is present in a range of 250 mg to 5000 mg; and,
biotin is present in a range of 250 μg to 10000 μg.

9. The method of claim 11, wherein the supplement daily dosage may be adjusted based on follow-up blood tests to optimize treatment outcomes.

10. A nutritional supplement composition for supporting neurological function in a patient, the composition comprising:
glycine in an amount between about 500 mg and about 5000 mg;
N-acetylcysteine in an amount between about 500 mg and about 5000 mg;
choline in an amount between about 250 mg and about 5000 mg; and,
biotin in an amount between about 250 mg and about 10000 μg.

11. The composition of claim 10, wherein the supplement is formulated for oral daily administration.

12. The composition of claim 10, wherein the supplement is administered as part of a personalized treatment protocol based on biomarker testing of the patient's blood.

13. The composition of claim 10, wherein the biomarkers comprise one or more of: inflammatory markers, oxidative stress indicators, or markers of neuronal injury.

14. The composition of claim 10, wherein the total daily dosage comprises:
about 1500 mg of glycine;
about 1500 mg of N-acetylcysteine;
about 1000 mg of choline; and,
about 10000 μg of biotin.

15. The composition of claim 10, wherein the supplement is used to treat or manage a neurological disorder selected from the group consisting of: neurodegenerative disease, traumatic brain injury, stroke, cognitive impairment, and neuroinflammation.
